(19) United States
(12) Patent Application Publication
(10) Pub. No.: US 2018/0211719 A1
(43) Pub. Date: Jul. 26, 2018

(54) SYSTEMATIC PHARMACOLOGICAL METHOD FOR PERSONALIZED MEDICINE

(71) Applicants: Wuhan Bio-links Technology Co., Ltd., Wuhan (CN); Huazhong Agricultural University, Wuhan (CN)

(72) Inventors: Hongyu Zhang, Wuhan (CN); Xionghui Zhou, Wuhan (CN); Yuan Quan, Wuhan (CN); Zejia Cui, Wuhan (CN); Qingyong Yang, Wuhan (CN)

(21) Appl. No.: 15/876,194

(22) Filed: Jan. 21, 2018

(30) Foreign Application Priority Data
Jan. 22, 2017 (CN) 201710046416.0

(51) Int. Cl. G16H 20/10; G06F 19/20; G16H 10/60

(52) U.S. Cl. CPC G16H 20/10; G16H 50/30; G16H 10/60; G06F 19/20

(57) ABSTRACT

The present invention discloses a systematic pharmacological method for personalized medicine. In the present invention, biological networks such as gene dependence networks are employed to reflect relationships between genes in pathogenesis. In combination with the gene expression data of a specific patient, a gene rank algorithm, which is capable of utilizing inter-genetic regulation relationships to mine key genes in the pathogenesis of disease in a specific patient, is used to construct a key genes list. Then, personalized medicine is carried out according to whether the drug targets are significantly targeted to the key genes list in the pathogenesis of disease in the specific patient. The systematic pharmacological method for personalized medicine proposed by the present invention is easy to implement, has low cost and high efficiency, and has wide application prospects in precision medicine and drug discovery.

### CROSS-REFERENCE TO PRIOR APPLICATION

This application claims the benefit of Chinese Patent Application No. 201710046416.0 filed on Jan. 22, 2017, the contents of which are incorporated herein by reference.

### FIELD OF THE INVENTION

The present invention relates to the biomedical technology field and, in particular, to a systematic pharmacological method for personalized medicine using gene expression data of patients and drug target data, based on biological networks.

### BACKGROUND OF THE INVENTION

Personalized medicine refers to choosing suitable drugs for a specific patient or patient population. It is a core target and an important topic for precision medicine (see: Collins F S, Varmus H. A new initiative on precision medicine [J]. N Engl J Med, 2015, 372(9): 793-795.; Dittmer J, Leyh B. The Impact of Tumor Stroma on Drug Response in Breast Cancer[J]. Seminars in Cancer Biology, 2015, 31: 3-15.). Current approaches mainly start with biological data of large samples to mine the mutated genes, in order to select drugs that target the genotypic variation according to the patient's genotype. However, such methods are of little clinical value because the penetrance of the mutated genes cannot be determined (see: AK M, JP I, IS K. Clinical Genomics: From Pathogenicity Claims to Quantitative Risk Estimates [J]. JAMA, 2016.); meanwhile, due to the general lack of interpretation of the disease mechanism in these kinds of identifications of genetic variations, these genes are usually not the true driver genes (see Burrell R A, McGranahan N, Bartek J, et al., The causes and consequences of genetic heterogeneity in cancer evolution [J]. Nature, 2013, 501(7467): 338-345.); furthermore, for a complex disease like cancer, there are usually more than one disease-causing gene, and the method of determining the corresponding drug based on a single genotype often does not lead to good treatment effects.

In a patent application previously filed by us (see the patent application “Pharmaceutical activity prediction and selection method based on genetic expressions and drug targets”, Hongyu Zhang, et al.), based on the assumption that gene expression data carry a wide range of modifications of genes such as genomic modifications and epigenetic modifications, and if a drug target significantly targets the differentially expressed gene of a patient, then the drug is the appropriate drug for this patient. The patent application used patients' gene expression data and drug target data to screen drugs for the patients, and certain effects were shown. This patent application shows the potential for using patient gene expression data and drug target data for personalized medicine. However, to the best of our knowledge, the truly decisive genes (driver genes) may not be those with a significant differential expression. Therefore, the methods which use differentially expressed genes as important genes in the pathogenesis of cancer are not sufficient for discovering all the key genes in the pathogenesis of cancer (see: Wagenblast E, Soto M, Gutierrez-Angel S, et al. a Model of Breast Cancer Heterogeneity Reveals Vascular Mimicry as a Driver of Metastasis [J]. Nature, 2015, 520 (7547): 358-362.). Biological networks are widely used in biological research as they can reflect the mutual regulation relationships between genes (see: Albert-LászlóBarabási, Zoltán N. Oltvai. Network Biology: Understanding the Cells' Functional Organization [J]. Nature Reviews Genetics, 2004. 5: 101-113.). However, how to use the appropriate biological networks to mine the key genes in the pathogenesis of a specific cancer patient and to carry out personalized medicine according to these key genes remains a problem to be solved.

### SUMMARY OF THE INVENTION

The objective of the present invention is to overcome the shortcomings of the prior art and to provide a systematic pharmacological method for personalized medicine. This method has a solid theoretical foundation, is easy to implement, has low cost and high efficiency, and has wide application prospects in precision medicine.

The technical solution of the present invention is: a systematic pharmacological method for personalized medicine, characterized in that it comprises the following steps:

Step 1: obtaining high-throughput biological data to build a biological network, the biological network describes interactions between genes;

Step 2: obtaining gene expression data of a diseased tissue of a specific patient suffering from the disease and the corresponding control data, calculating a differential expression value for genes of the specific patient accordingly;

Step 3: sorting genes of the specific patient according to their importance using a gene rank algorithm according to the biological network and the differential expression value of genes of the specific patient, selecting a plurality of top-ranked, more important genes as key genes to construct a key genes list;

Step 4: obtaining a target gene corresponding to a drug to be tested according to information in a drug target database; and

Step 5: using statistical analysis to check whether the target gene of the drug to be tested is significantly targeted to the key genes list in order to predict the activity of the drug on the specific patient; screening a drug suitable for the specific patient to achieve personalized medicine for the specific patient.

In the present invention, biological networks are employed to reflect the relationships between genes in pathogenesis. In combination with the gene expression data of a specific patient, a gene rank algorithm which is capable of utilizing the inter-genetic regulation relationships to mine key genes in the pathogenesis of disease in a specific patient is used, in order to achieve personalized medicine according to whether the drug targets are significantly targeted to the key genes list in the pathogenesis of disease in the specific patient.

In the technical solution above, the biological network is a directed network or an undirected network; the directed network is a gene dependency network, the undirected network is a protein interaction network or a gene co-expression network. The vertices in the undirected network are genes, and the edge between two vertices indicates an interaction between two genes. The gene dependency network refers to a gene dependency network related to disease phenotypes, it is a directed network, and includes vertices and directed edges connecting two vertices, wherein a vertex is a gene, a directed edge between two vertices denotes that the relationship between one of the genes (the gene represented by the vertex corresponding to the start point of the directed edge, i.e., the start-point gene) and the disease phenotype is dependent on the other gene (the gene represented by the vertex corresponding to the end point of the directed edge, i.e. the end-point gene) pointed to by the arrow. Since phenotypic information is incorporated during the construction of the gene dependency network, the gene dependency network could reveal the presence of gene dependency relationships during specific phenotypic changes.

In the present invention, personalized medicine of a specific patient can be achieved if drug activity predictions are carried out for this specific patient. If precise medication is to be carried out for a specific patient group (such as a certain subtype of cancer) of the disease, it can be achieved by screening drugs (or drug combinations) that have a higher proportion of drug activity in the patient population with this subtype.

In the present invention, the drug to be tested may be a single composition drug or a combination drug. When the drug to be tested is a single composition drug, said target gene is a target corresponding to the drug; when the drug to be tested is a combination drug, said target gene is the union set of the target genes corresponding to each drug of the combination drug.

As a further improvement of the above technical solution, the gene dependency network is constructed by obtaining gene expression data and the corresponding clinical phenotype data for diseased tissues of a plurality of patient samples corresponding to the disease, and comprises the following steps:

Step A. using the gene expression data and the corresponding clinical phenotype data, obtaining a gene dependency index between any two genes; and

Step B. setting a threshold value, obtaining significantly dependent gene pairs, constructing all the significantly dependent gene pairs into the gene dependency network;

in the gene dependency network, a vertex is a gene, a directed edge between two vertices indicates the presence of a significant dependency relationship between an end-point gene and a relationship between a start-point gene and the disease phenotype. The methods for the selection of thresholds could be: selecting a specific percentage, statistical inference for indexes or rearranging of the expression profile to construct a random background to obtain a significance threshold.

As a further improvement of the above technical solution, the protein interaction network is constructed by obtaining protein interaction pairs with high confidence from a protein interaction database, and comprises the following steps:

Step I. downloading human protein interaction data from the protein interaction database; and

Step II. screening the protein interaction pairs with high confidence, and constructing all the protein interaction pairs with high confidence as the protein interaction network;

in the protein interaction network, a vertex is a gene, an edge between two vertices indicates interaction between two proteins corresponding to the two vertices.

The protein interaction database can be any protein interaction database used in the art, for example, the STRING database.

As a further improvement of the above technical solution, the gene-dependency index is conditional mutual information. If the relevance between a gene A and the patient's phenotype P (e.g., metastasis or not) significantly depends on another gene B, then there is a gene dependency relationship (A→B) between the two genes. When calculating the gene dependency relationship (i.e., conditional mutual information), CMI (A,P/B) is calculated, which denotes the dependency relationship between the mutual information between the expression value of gene A and phenotype P and the expression value of gene B, or the mutual information value between A and P in the condition of gene B. All the gene dependency relationship pairs constitute a gene dependency network. A method for constructing gene dependency networks can also be found in Zhou X, Liu J. Inferring Gene Dependency Network Specific to Phenotypic Alteration Based on Gene Expression Data and Clinical Information of Breast Cancer [J]. PLoS ONE, 2014, 9(3): e92023.

As a further improvement of the above technical solution, the conditional mutual information is calculated as follows:

CMI(A,P|B)=I_{high}(A,P)−I_{low}(A,P)

where CMI (A,P/B) denotes mutual information between gene A and disease phenotype P under the condition of gene B; I_{high}(A,P) denotes mutual information between the expression value of gene A and disease phenotype data in patient samples with highly expressed gene B; I_{low}(A,P) denotes mutual information between the expression value of gene A and disease phenotype data in patient samples with lowly expressed gene B;

the mutual information is calculated as follows:

$$I ( A , P ) = {\sum }_{A} {\sum }_{P} p ( A , P ) {log}_{2} \frac{p ( A , P )}{p ( A ) p ( P )}$$

where p(A) is the probability of variable A, p(A,P) is the joint probability of variable A with variable P, variable A is the expression value of gene A, variable P is disease phenotype P.

As a further improvement of the above technical solution, the gene expression data is obtained by a gene expression analysis method, the gene expression analysis method includes at least one of gene chip or RNA-Seq, the differential expression value for genes of the specific patient is obtained by fold-change. The differential expression value is the absolute value of fold-change.

As a further improvement of the above technical solution, the gene rank algorithm is a revised PageRank algorithm, the formula of the revised PageRank algorithm is as follows:

$${r}_{j}^{n} = ( 1 - d ) {ex}_{j} + d {\sum }_{i = 1}^{N} \frac{{w}_{ij} {r}_{i}^{n - 1}}{{deg}_{i}}$$

Where rⱼⁿ is a calculated importance value of gene j, exⱼ is an initial value of gene j, and wᵢⱼ is a relationship between genes in the biological network; when the biological network is a directed network, if gene i depends on gene j, then wᵢⱼ=1, otherwise wᵢⱼ=0; when the biological network is an undirected network, if there is interaction between gene i and gene j, then wᵢⱼ=1 and wⱼᵢ=1, otherwise wᵢⱼ=0 and wⱼᵢ=0; rᵢⁿ⁻¹ is a value of the i^{th} gene after the (n−1)^{th} iteration; deg, is an out-degree of the vertex i; parameter d (0≤d<1) is a constant and d denotes the proportion of the biological network during calculation. In this algorithm, the input is a matrix describing the relationships between genes and a vector containing the differential expression value of genes, and the output is the importance value of each gene in the pathogenesis of disease in the patient. The gene rank algorithm can also be found in more detail in Morrison J L, Breitling R, Higham D J, et al. GeneRank: Using Search Engine Technology for the Analysis of Microarray Experiments [J]. BMC Bioinformatics, 2005, 6(1): 1.

As a further improvement of the above technical solution, the drug target database is at least one of DGIdb (Drug-Gene Interaction database), TTD (Therapeutic Target Database) or Drugbank.

As a further improvement of the above technical solution, the drug target database is DGIdb (Drug-Gene Interaction database), TTD (Therapeutic Target Database) and Drugbank, the target gene corresponding to a drug to be tested is a union set of target gene data of three databases including DGIdb, TTD and Drugbank.

As a further improvement of the above technical solution, the statistical analysis is enrichment analysis; by analyzing whether the target gene corresponding to a drug to be tested is enriched in the key genes list, whether it significantly targets the important genes list is determined.

As a further improvement of the above technical solution, an enrichment analysis model used in the enrichment analysis is the Kolmogorov-Smirnov test.

As a further improvement of the above technical solution, the disease is cancer, the diseased tissue is cancer tissue, and the control data is the gene expression data of paracarcinoma tissue or normal tissue.

As a further improvement of the above technical solution, the diseased tissue is cancer tissue, the control data is the gene expression data of paracarcinoma tissue.

The present invention also provides application of the systematic pharmacological method for personalized medicine in the screening of personalized drugs and/or drug combinations, and in personalized medicine.

Compared with the prior art, the beneficial effects of the present invention are as follows:

The present invention provides a personalized medicine method based on biological networks and gene expression profiling, which is easy to implement, has high efficiency and a wide application range. The method of the present invention can be used in the screening of suitable drugs (including drug combinations) for individual patients, so as to provide personalized treatment for this patient. The invention can also be used for precise medication of patients with subtypes of certain diseases. The invention has broad application prospects in the field of precision medicine.

### BRIEF DESCRIPTION OF THE DRAWINGS

FIG. 1 is a flow diagram of an embodiment of a systematic pharmacological method for personalized medicine of the present invention.

FIG. 2 is a flow scheme of an embodiment of a systematic pharmacological method for personalized medicine of the present invention (taking a gene dependency network as an example of a biological network).

FIG. 3 is a graph showing the survival analysis of ovarian cancer patients in embodiment 1 of the present invention. The abscissa is the survival time and the ordinate is the survival rate (i.e., the proportion of patient samples that are still alive in the current group of patients); among all the patients, the top 30% of patients that received the most appropriate medication were divided into a correctly treated group and the other patients were divided into a wrongly treated group; truncated data refers to samples that are not dead at the current time of tracking; hazard ratio refers to the ratio of death risk of the two groups of patients, 1.32 in the present figure refers to the fact that patients in the wrongly treated group had a risk of death that was 1.32 times of that of the patients in the correctly treated group; p-value is the statistical significance of the difference in risk of death between the two groups of patients (log-rank test).

FIG. 4 is a graph showing the survival curve of glioblastoma multiforme of embodiment 2. The abscissa is the survival time, the ordinate is the survival rate (i.e., the proportion of patient samples that are still alive in the current group of patients); among all the patients, the top 30% of patients that received the most appropriate medication were divided into a correctly treated group and the other patients were divided into a wrongly treated group; truncated data refers to samples that are not dead at the current time of tracking; hazard ratio refers to the ratio of death risk of the two groups of patients, 1.46 in the present figure refers to the fact that patients in the wrongly treated group had a risk of death that was 1.46 times of that of the patients in the correctly treated group; p-value is the statistical significance of the difference in risk of death between the two groups of patients (log-rank test).

FIG. 5 is a graph showing the survival curve of breast cancer of embodiment 3. The abscissa is the survival time, the ordinate is the survival rate (i.e., the proportion of patient samples that are still alive in the current group of patients); among all the patients, the top 30% of patients who received the most appropriate medication were divided into a correctly treated group and the other patients were divided into a wrongly treated group; truncated data refers to samples that are not dead at the current time of tracking; hazard ratio refers to the ratio of death risk of the two groups of patients, 1.86 in the present figure refers to the fact that patients in the wrong treatment group had a risk of death that was 1.86 times of that of the patients in the correctly treated group; p-value is the statistical significance of the difference in risk of death between the two groups of patients (log-rank test).

FIG. 6 is a comparison of the effectiveness of listed drugs and clinical drugs in patients with breast cancer in embodiment 4 of the present invention. The abscissa is the drug's activity on the samples; p-value is the statistical significance of a drug (combination) targets towards the key genes list of a patient with the Kolmogorov-Smirnov test; the smaller the p-value, the stronger the activity.

### DETAILED DESCRIPTION OF THE ILLUSTRATED EMBODIMENTS

The essence of drug therapy is the interaction of drugs (small molecules) with virulence factors (targets), thereby regulating the expression and/or function of disease-related genes. However, it is difficult to mine personalized disease-causing genes for a particular patient using traditional methods. Here we propose a method for personalized medicine based on biological networks, gene expression profiling of patients and drug target data.

The present method predicts activities of drugs (or drug combinations) by analyzing whether a target gene of a drug (or a target gene set of a drug combination) targets key genes in the pathogenesis of disease in patients, thereby determining an appropriate therapeutic drug. In order to mine the key genes in the pathogenesis of patients, biological networks are used to depict relationships between genes in the pathogenesis of diseases. These networks are based on the assumption that if a number of genes have a relationship with a certain gene, then this gene is likely to be a key gene. The differential expression value of a gene is employed as the initial importance of each gene and a modified Pagerank algorithm is used to mine the key genes in the pathogenesis of disease in patients in the present invention. A feature of this invention is that it starts from basic principles of drug action, utilizes relationships between genes contained in gene expression profiling of the patient population and personalized information contained in gene expression profiling of a specific patient, and does not rely on genetic models, thereby achieving rational personalized treatment. The present invention has broad application potential.

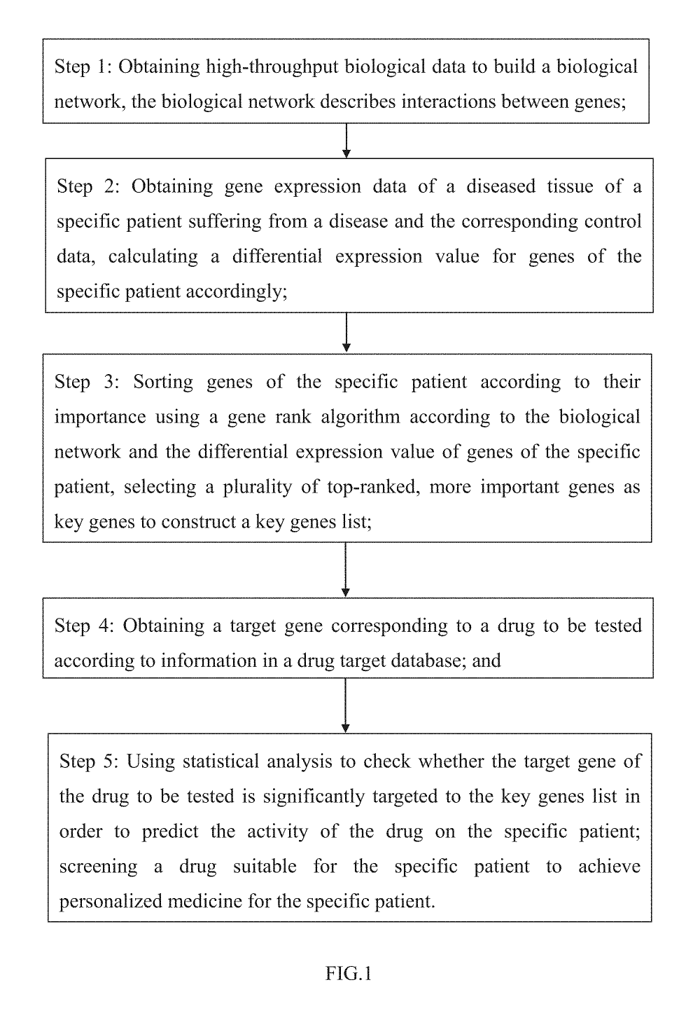

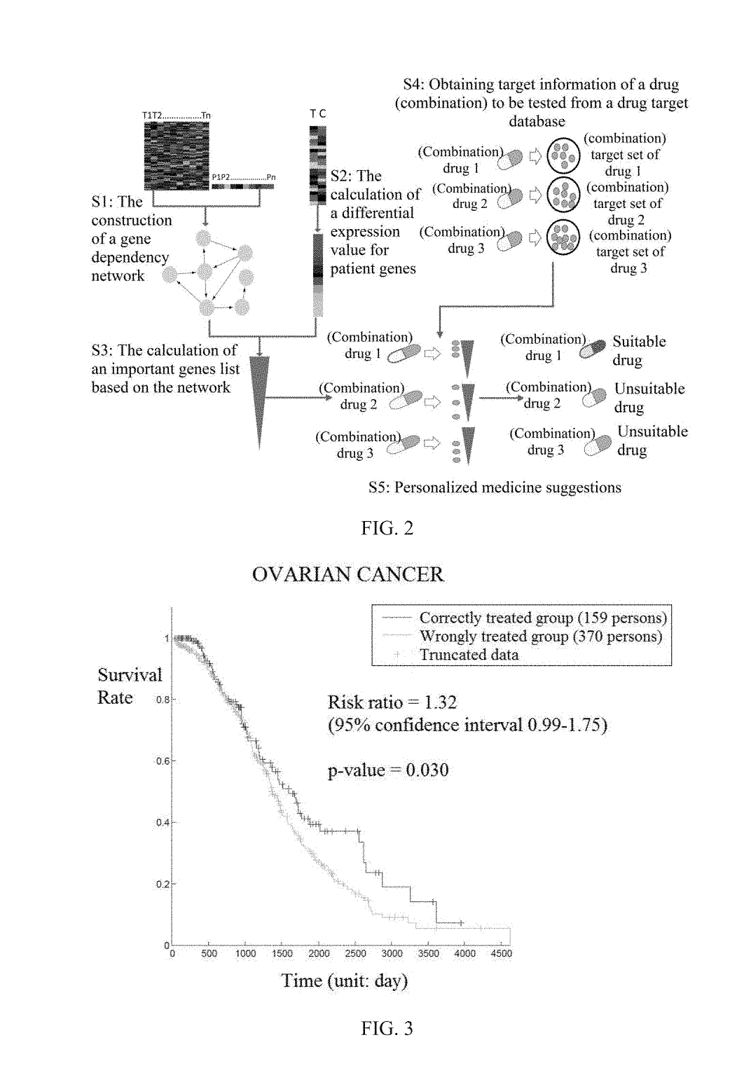

FIGS. 1 and 2 are a flow diagram and a flow scheme of an embodiment of a systematic pharmacological method for personalized medicine. The present invention utilizes gene expression data and drug target data of the patient population of a certain disease, performs appropriate drug screening on a specific patient or patient population based on a biological network, thereby achieving personalized medicine. The method of the present invention comprises the steps of:

Step 1: obtaining high-throughput biological data to build a biological network, the biological network describes interactions between genes; the biological network may be a directed network such as a gene dependency network, or it may be an undirected network, such as a protein interaction network or a gene co-expression network.

When choosing the protein interaction network to describe relationships between genes, human protein interaction data with high confidence can be downloaded from databases such as the STRING database (Szklarczyk D, Franceschini A, Wyder S, et al. STRING v10: protein-protein interaction networks, integrated over the tree of life[J]. Nucleic Acids Research, 2015, 43 (Database issue): D447). Then, these high-intensity interaction pairs (e.g., the intensity of the interaction being not less than 400) is constructed as a protein interaction network.

When choosing the gene dependency networks to describe relationships between genes, the gene dependency network needs to be constructed based on gene expression data of patient populations consist of a plurality of patient samples and the corresponding clinical data, wherein the clinical phenotype data could be selected according to the disease type. When the disease is cancer, the clinical phenotype data may be the patient's death time (i.e., from the time of diagnosis of the patient to the latest tracked time) and the state of death. Patient samples with clear prognostic information, that is, those that allow accurate determination of prognosis status (such as poor prognosis or good prognosis), are usually selected. The patient samples typically include patient samples of different disease phenotypes. For example, when the disease is cancer, the patient samples include patient samples of different death times.

Said gene dependency networks reflect gene dependency relationships in disease pathogenesis. If the relevance between a gene A and the patient's phenotype P (e.g., metastasis or not) significantly depends on another gene B, then there is a gene dependency relationship (A→B) between the two genes, and all the pairs with gene dependency relationships constitute a gene dependency network. In a preferred embodiment, the construction of a gene dependency network comprises the following steps: using the gene expression data and the corresponding clinical phenotype data, obtaining a gene dependency index between any two genes; setting a threshold value, obtaining significantly dependent gene pairs, constructing all the significantly dependent gene pairs into a gene dependency network; in the gene dependency network, a vertex is a gene, a directed edge between two vertices indicates the presence of a significant dependency relationship between an end-point gene and a relationship between a start-point gene and the disease phenotype. Moreover, the gene-dependency index is conditional mutual information, and the conditional mutual information is calculated as follows:

CMI(A,P|B)=I_{high}(A,P)−I_{low}(A,P)

Where CMI (A,P/B) denotes the mutual information between gene A and disease phenotype P under the condition of gene B; I_{high}(A,P) denotes the mutual information of the expression value of gene A and disease phenotype data in patient samples with highly expressed gene B; I_{low}(A,P) denotes the mutual information of the expression value of gene A and disease phenotype data in patient samples with lowly expressed gene B. The expression value of gene A used in the calculation is usually the data after discretization of the expression data of gene A acquired, and the disease phenotype value is the data after discretization of the clinical phenotype data acquired. In a preferred embodiment, the discretization method for the expression data of gene A is: setting the median of the expression data of gene A in all patient samples as a base, if the expression data of gene A of a certain patient sample is smaller than or equal to the median, the expression value of gene A of this patient sample is set to 0, otherwise it is set to 1. In a preferred embodiment, the discretization method for clinical phenotypic data is: selecting an appropriate discretization criteria based on the clinical phenotype data obtained, dividing the clinical phenotype data into two groups, setting the disease phenotype data of one group of patient samples with relatively poor prognosis to 0, setting the disease phenotype data of another group of patient samples with relatively good prognosis to 1.

The mutual information is calculated as follows:

$$I ( A , P ) = {\sum }_{A} {\sum }_{P} p ( A , P ) {log}_{2} \frac{p ( A , P )}{p ( A ) p ( P )}$$

Where p(A) is the probability of a variable A, p(A,P) is the joint probability of the variable A with a variable P, the variable A is the expression value of gene A, the variable P is the disease phenotype P.

A method for constructing gene dependency networks can also be found in: Zhou X, Liu J. Inferring Gene Dependency Network Specific to Phenotypic Alteration Based on Gene Expression Data and Clinical Information of Breast Cancer [J]. PLoS ONE, 2014, 9(3): e92023.

The gene expression data is obtained by a gene expression analysis method, said gene expression analysis method includes at least one of gene chip or RNA-Seq. In a preferred embodiment of the present invention, the gene expression data includes gene expression data obtained by gene chip and RNA-Seq.

Step 2: obtaining gene expression data of a diseased tissue of a specific patient suffering from the disease and the corresponding control data, calculating a differential expression value for genes of the specific patient accordingly.

Here, the control data is the gene expression data of normal tissues corresponding to diseased tissues. In a preferred embodiment of the present invention, the diseased tissue is cancer tissue; the control tissue is paracarcinoma tissue or normal tissue. As paracarcinoma tissue and cancer tissue have similar tumor microenvironments, the gene expression data of paracarcinoma tissues is more preferred.

According to the gene expression data of diseased tissue and the corresponding control data, the differential expression values of the genes of the specific patient can be calculated using statistical tests (such as t-test and chi-square test), bioinformatics methods (such as fold-change, SAM) or machine learning methods. In a preferred embodiment of the present invention, differential expression values of the specific patient's genes are obtained by fold-change, that is, the absolute value of fold-change for each gene is calculated. Fold-change is calculated as follows:

fd=log₂^{a}−log₂^{b}

where a is the expression value of a gene in the diseased tissue of the patient, b is the expression value of the gene in the control tissue. Therefore, when fd>0, it indicates that the gene is up-regulated by fd times in diseased tissues, and if fd<0, the gene is down-regulated by fd times.

Step 3: sorting genes of the specific patient according to their importance using a gene rank algorithm according to the biological network and the differential expression value of genes of the specific patient, selecting a plurality of top-ranked, more important genes as key genes to construct a key genes list.

Here, we use the absolute value of the differential expression value as the initial importance of the genes of the patient; a gene rank algorithm is used to calculate a key genes list in the pathogenesis of the disease in patients. The input of the algorithm is a matrix composed of relationships between the genes and the initial importance of each gene. If a gene is pointed (dependent) by many important genes, then the gene is more important.

A preferred algorithm of the gene rank algorithm is a revised PageRank algorithm. The core idea of this algorithm is that the importance of a vertex in a network depends on the importance of the vertex pointing towards it. The formula of the algorithm is as follows:

$${r}_{j}^{n} = ( 1 - d ) {ex}_{j} + d {\sum }_{i = 1}^{N} \frac{{w}_{ij} {r}_{i}^{n - 1}}{{deg}_{i}}$$

Where rⱼⁿ is a calculated importance value of gene j, exⱼ is an initial value of gene j, and wᵢⱼ is a relationship between genes in the biological network; when the biological network is a directed network, if gene i depends on gene j, then wᵢⱼ=1, otherwise wᵢⱼ=0; when the biological network is an undirected network, such as a protein interaction network, if there is an interaction between gene i and gene j, then wᵢⱼ=1 and wⱼᵢ=1, otherwise wᵢⱼ=0 and wⱼᵢ=0; rᵢⁿ⁻¹ is a value of the i^{th} gene after the (n−1)^{th} iteration; deg, is an out-degree of the vertex i; parameter d (0≤d<1) is a constant and d denotes the proportion of the biological network during calculation. From the formula, it is understood that the importance of vertex j depends on the values of two parts: the initial importance of the gene (exⱼ in the formula, the differential expression value of gene i in the present invention) and the importance values of all the vertices pointing to vertex j (the second term to the right of the formula). The larger the parameter d is, the greater the importance of the gene is dependent on the relationship between the genes; the smaller the parameter d is, the greater the importance of the gene is dependent on the initial importance of the gene. In the present invention, d is preferentially chosen to be 0.5, and the algorithm iterates only once.

Step 4: obtaining a target gene corresponding to the drug to be tested according to information in a drug target database.

Here, the drug to be tested may be a single composition drug or a combination drug. When the drug to be tested is a single composition drug, said target gene is a target of the drug; when the drug to be tested is a combination drug, the target gene is the union set of the target genes corresponding to each drug of the combination drug.

The drug target database is at least one of DGIdb, TTD or Drugbank; more preferably, the drug target database is DGIdb, TTD and Drugbank, and the target gene of the drug to be tested is a union set of target gene data of three databases including DGIdb, TTD and Drugbank. This will make the prediction more accurate.

Step 5: using statistical analysis to check whether the target gene of the drug to be tested is significantly targeted to the key genes list to predict the activity of the drug on the specific patient, screening a drug suitable for the specific patient to achieve personalized medication for the specific patient.

The statistical analysis refers to enrichment analysis. By analyzing whether the target gene corresponding to the drug to be tested obtained in Step 4 is enriched in the key genes list in the pathogenesis of disease in the patient, whether it targets the important genes list (key genes list) in the pathogenesis of disease in the patient is determined; if the target genes of a drug is enriched in the key genes list in the pathogenesis of disease in a particular patient, then the drug is suitable for the patient. In particular, when the drug is a combination drug, if a set of the target genes of the combination drug (i.e., a union set of the target gene data of these several drugs) is enriched in the key genes list in the pathogenesis of disease in the specific patient, then these drugs are a suitable combination drug for the patient.

The line of thinking behind drug target gene enrichment analysis is that for a certain drug or combination drug, if the target genes of this drug or combination drug is mainly distributed on the upper part of the key genes list (important genes set) of the patient, it is thought that the drug or drug combination has good therapeutic effects on this patient. A specific method is to use the Kolmogorov-Smirnov test, which is able to test whether the elements in a set are significantly distributed in either the upper or lower part of a sequence. In the present invention, genes located in the upper part of the genes list are the most important genes. Therefore, we use this statistical method to see if target genes of a (combination) drug are significantly distributed on the upper part of the key genes list of the patient. In this way, based on the drug target gene dataset obtained in Step 4 and the key genes list (i.e., the important genes list) in the pathogenesis of disease in the patient obtained in Step 3, we can use the enrichment analysis in Step 5 to see if the target genes of the drug mainly target important genes in the pathogenesis of disease in the patient, thereby predicting whether the drug has therapeutic effect on the patient. For the Kolmogorov-Smirnov test, it is thought that the smaller the p-value of the test is, the more appropriate the drug is for this patient. During the construction of the efficacy prediction model for drugs on a specific patient, no class labels are used and no training process is involved.

The present invention integrates and uses gene expression data (including gene expression data obtained by gene expression analysis methods such as gene chip and RNA-Seq) and drug target information. In the present invention, drug target gene information and the information of key pathogenic genes of the patient could not only be used to predict the efficacy of certain drugs or the combination of some drugs (i.e. drug combinations) on the patient, but also to screen out the most suitable drug (or drug combination) for the patient, including drugs that are not currently used for this disease. Therefore, the method of the present invention could not only predict the efficacy of a particular drug (combination) on a certain patient, but also achieve drug (or drug combination) screening, or even drug repositioning.

For patients with any disease, as long as gene expression data and clinical data of the patient population with this disease could be obtained in a public database, and the gene expression data and the corresponding control data of the patient to undergo personalized medicine are obtained, drugs (or drug combinations) suitable for this patient can be screened out, thereby achieving personalized medicine for this patient. In the meantime, more practically, for a particular patient population (such as patient population with a certain subtype of cancer), we only have to obtain the gene expression data of the patient population with this disease, the clinical phenotype data of the patients and the subtype information of the patients in a public database to screen out drugs which have obvious preferences for a specific subtype (the efficacies of these drugs or drug combinations on this subtype are significantly higher than on other subtypes), thereby achieving precise medication for the population with this subtype of the disease. As a result, our method is easy to implement, efficient and can be used extensively for precise medication of specific patients and patient populations.

To better illustrate the objectives, technical solutions and advantages of the present invention, the present invention will be further described with reference to the accompanying drawings and specific embodiments.

In the embodiments, the experimental methods used are conventional methods unless otherwise specified. Materials, reagents and the like used are commercially available unless otherwise specified. The clinical data in the embodiment is clinical phenotypic data.

### Embodiment 1: Using the Method of the Present Invention for Personalized Medicine of Ovarian Cancer Patients and Efficacy Validation

I. Obtaining Gene Expression Data of Ovarian Cancer Patient Samples, Gene Expression Data of Control Samples, and Clinical Data of Patients

Gene expression data (level 3 data from Agilent G4502A chip) of ovarian cancer patient samples, clinical data (death time, death status) of these patients and gene expression data of control samples were obtained. Missing clinical data or missing medication information (or the number of drug targets being too small to be suitable for statistical analysis; in the present invention, a screening criterion was set at no less than 10 targets) were deleted, and gene expression data and prognostic data of 584 patients were obtained. The data of these 584 patients were used in the construction of the gene dependency network of step 2. Among the 584 patients, 529 cancer patients contained medication information (and drug targets met our criteria). Gene expression data and medication data of these 529 patients and gene expression data of 8 normal ovarian tissue samples could be used for the validation of drug efficacy.

II. Calculating the Key Genes List in the Pathogenesis of Disease for Each Patient

The following procedures were followed in order to calculate the key genes list in the pathogenesis of disease in each patient:

A. Gene dependency networks were constructed based on gene expression data and clinical data of 584 ovarian cancer patients.

B. The expression value of each gene and patient clinical data were discretized. The discretization method of gene expression data was: the median of the expression data of a gene in all patient samples was set as a base; if the expression data of this gene in a certain sample was smaller than or equal to the median, the expression value was set to 0, otherwise it was set to 1. For the clinical data of cancer patients, if their death time is no less than 1200 days, the expression value is set to 0; if their death time is less than 1200 days and their death status is “dead”, the expression value is set to 1. Other patients were in an intermediate status and were not used for the construction of gene dependency networks.

C. 100000 pairs of genes were randomly selected, the gene dependency value between each pair of genes was calculated, and the 100000 gene dependency values were used as background distribution. The gene dependent value of the mutual information between gene A and phenotype P (clinical data after discretization) relative to gene B was calculated as follows:

\textcircled{1} The variables A, P, B were organized into triples (N×3, N is the number of cancer samples involved in the calculation), they were sorted according to the expression levels of gene B from small to large.

\textcircled{2} The top-ranked 35% of the triples were selected, and the mutual information between gene A and phenotype P was calculated (for the calculation of mutual information, see Hanchuan Peng, Fuhui Long, and Chris Ding, “Feature Selection Based on Mutual Information: Criteria of Max-dependency, Max-relevance, and Min-redundancy,” IEEE Transactions on Pattern Analysis and Machine Intelligence [J], Vol. 27, No. 8, pp. 1226-1238, 2005.), denoted as I_{low}. The bottom-ranked 35% of the triples were selected, and the mutual information between gene A and phenotype P was calculated and denoted as I_{high}. The difference between I_{high} and I_{low} was the gene dependency value of the mutual information between gene A and phenotype P (correlation) relative to gene B.

D. For the 17788 genes gathered in the ovarian cancer data set, gene dependency values of all possible combinations of gene pairs were calculated. According to the background distribution obtained in step \textcircled{2}, the p-value of each pair of gene dependency relationship was estimated. Gene dependency relationship pairs with p-value<=10e-05 were preserved. All gene pairs with significant gene dependency relationships constituted a gene dependency network.

E. For each cancer patient, the fold-change value of each gene was calculated based on its gene expression data of the diseased tissue and control data (in the present embodiment, the average value of the gene expression data of 8 normal tissues was used as the gene expression data of the control sample. In practice, the gene expression data of the paracarcinoma tissue of each cancer patient was preferably used as the control). The absolute value of the fold-change value was used as the differential expression value (initial importance) of the gene.

F. The importance calculation of genes in the gene dependency network was carried out using a modified PageRank algorithm. The input of the modified PageRank algorithm was a matrix of gene dependency relationships and a vector containing the initial importance value (the absolute value of fold-change of the vertex). Finally, all genes of this patient were arranged in descending order of importance.

III. Summarizing the Drugs Used by the Patients and the Targets of these Drugs

Drug target databases (including DGIdb: http://dgidb.genome.wustl.edu/, DrugBank: http://www.drugbank.ca/ and TTD: http://bidd.nus.edu.sg/group/ttd/ttd.asp) were used in order to retrieve all drugs contained in the databases and their corresponding target data.

IV. Predicting Drug Efficacy for Each Patient

For each patient, in step one, we checked which drugs had been used by the patient; for each drug, we searched for its target gene in step three and grouped all the target genes of that drug into a set of target genes. The Kolmogorov-Smirnov test was used to see if the target genes of the drug were significantly distributed at the top of the key genes list obtained in step II, that is, whether the target genes of the drug were targeted to the most important genes in the pathogenesis of disease in the patient. The smaller the statistical p-value was, the more suitable the drug was for this patient.

V. Performance Evaluation of the Prediction

For the 529 ovarian cancer patients of this embodiment, if a plurality of drugs were used, the efficacy of the drug with the smallest p-value was taken as the efficacy in the patient. Then, the patients could be divided into two groups according to the efficacies of the drugs (p-value) in these patients. Survival analysis was performed on these two groups of patients based on their prognostic information (time of death, state of death) to see if patients in the group of better efficacy (30% of the patients with the lowest p-value) have a longer survival time than those in the group of worse efficacy (70% of the patient with the largest p-value). If the correctly treated group has a significantly longer survival time than the wrongly treated group, then the correctness of our drug efficacy prediction method can be confirmed.

In these 529 patients, survival analysis of both groups of patients showed that their hazard ratio was 1.31, the p-value of the log-rank test (the log-rank test is a test analyzing the survival time distribution of two groups of patients. The smaller the p-value, the greater the difference in survival time between the two groups) was 0.030, which indicated the effectiveness of our prediction method. The survival curves of the two groups of patients obtained in embodiment 1 were shown in FIG. 3.

### Embodiment 2: Use the Method of the Present Invention for Personalized Medicine of Glioblastoma Multiforme Patients and Efficacy Validation

I. Obtaining Gene Expression Data of Glioblastoma Multiforme Patient Samples, Gene Expression Data of Control Samples, and Clinical Data of Patients

Method for obtaining glioblastoma multiforme patient data from TCGA was the same as step I of embodiment 1, the gene expression data of 577 cancer patients and 10 normal tissue samples, as well as the prognosis information and medication information of these patients were obtained. Among these 574 patients, a total of 136 patients had medication information in line with standards. These patients could be used to validate the personalized medicine of patients.

II. Calculating the Key Genes List in the Pathogenesis of Disease in Patients

The key genes list of each glioblastoma multiforme patient (ranked in descending order of importance) was calculated using the same method as step II of embodiment 1. The difference was that when carrying out the discretization of the clinical data of glioblastoma multiforme patients, the threshold of death time was 400 days (256 people were set to 1, 261 people were set to 0).

III. Summarizing the Drugs Used by the Patients and the Targets of these Drugs

This step was the same as step III of embodiment 1.

IV. Predicting Drug Efficacy for Each Patient

This step was the same as step IV of embodiment 1.

V. Performance Evaluation of the Prediction

This step was the same as step V of embodiment 1.

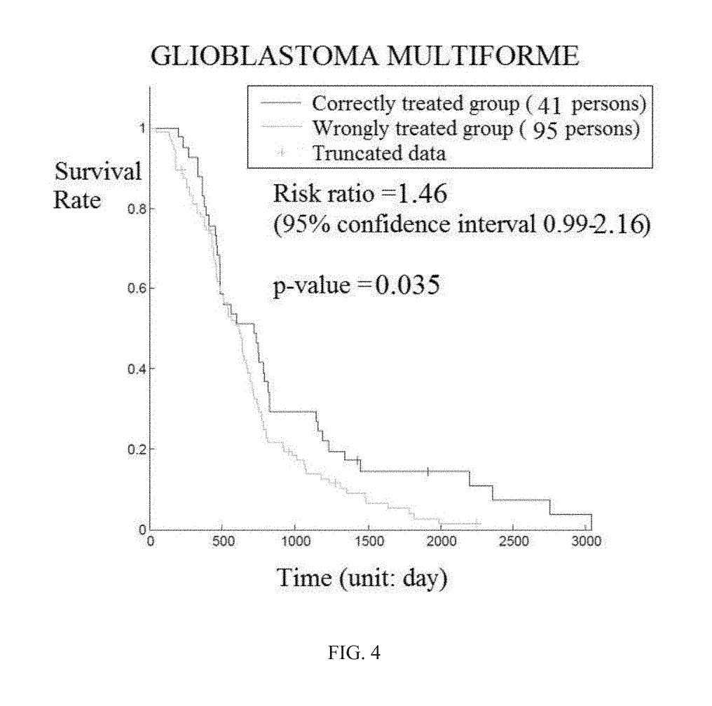

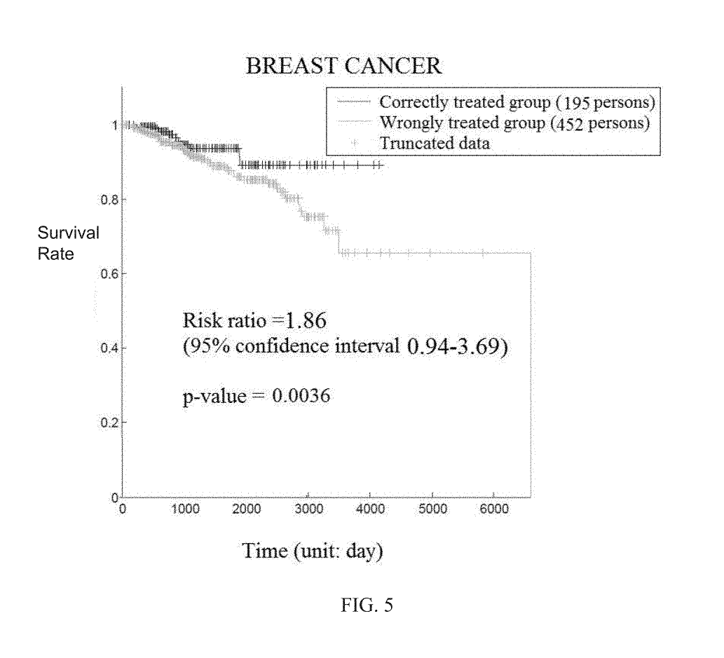

The 136 glioblastoma multiforme patients of this embodiment were divided into two groups according to the efficacies of the drugs in these patients (p-value). Survival analysis was performed on these two groups, the hazard ratio was shown to be 1.45, and the p-value of the log-rank test was 0.035. These results confirmed the effectiveness of our prediction method. The survival curves of the two groups of patients obtained in embodiment 2 were shown in FIG. 4.

### Embodiment 3: Use the Method of the Present Invention for Personalized Medicine of Breast Cancer Patients and Efficacy Validation

I. Obtaining Gene Expression Data of Breast Cancer Patient Samples, Gene Expression Data of Control Samples, and Clinical Data of Patients

Method for obtaining breast cancer patient data from TCGA was the same as step I of embodiment 1, the difference was that in this embodiment, the gene expression data was RNA-Seq data. The gene expression data of 1109 cancer patients and 113 normal tissue samples, as well as the prognosis information and medication information of these patients, were obtained. Among these 1109 patients, a total of 647 patients had medication information in line with standards. These patients could be used to validate the personalized medicine of patients.

II. Calculating the Key Genes List in the Pathogenesis of Disease in Patients.

The key genes list of each breast cancer patient (ranked in descending order of importance) was calculated using the same method as step II of embodiment 1. The difference was that when carrying out the discretization of the clinical data of breast cancer patients, the threshold of death time was 5 years (103 people were set to 1, 253 people were set to 0).

III. Summarizing the Drugs Used by the Patients and the Targets of these Drugs

This step was the same as step III of embodiment 1.

IV. Predicting Drug Efficacy for Each Patient

This step was the same as step IV of embodiment 1.

V. Performance Evaluation of the Prediction

This step was the same as step V of embodiment 1.

The 647 breast cancer patients of this embodiment were divided into two groups according to the efficacies of the drugs (p-value) in these patients. Survival analysis was performed on these two groups, the hazard ratio was shown to be 1.86, and the p-value of the log-rank test was 0.0036. These results confirmed the effectiveness of our prediction method. The survival curves of the two groups of patients obtained in embodiment 3 were shown in FIG. 5.

### Embodiment 4: Use the Method of the Present Invention for Personalized Medicine Screening for Patients with Alzheimer's Disease

I. Obtaining Gene Expression Data of Alzheimer's Disease Patient Samples and Control Samples

The chip data (series: GSE5281) of patients with Alzheimer's disease and their controls were downloaded from the GEO Dataset database to generate gene expression data of 87 brain samples of Alzheimer's disease patients and 74 control samples.

II. Calculating the Key Genes List of Patients with Alzheimer's Disease

Based on the gene names in the 161 chip data downloaded in step I and the human protein interaction data downloaded from the STRING database (http://string-db.org/), interaction pairs which existed in humans and had physical interaction of no less than 400 were selected to construct a gene interaction matrix; the fold-change value of each gene for each patient was calculated as an initial importance to input into the GeneRank algorithm and key genes lists of patients with Alzheimer's disease and of the general population were calculated. Finally, all genes in this patient were arranged in descending order of importance.

III. Summarizing Alzheimer's Disease Drugs that are Currently Under Development or Successfully Listed and their Targets.

Drug target databases (including DGIdb: http://dgidb.genome.wustl.edu/, DrugBank: http://www.drugbank.ca/ and TTD: http://bidd.nus.edu.sg/group/ttd/ttd.asp) were searched in order to retrieve all the Alzheimer's disease drugs that were successfully listed (83 drugs) and currently under development (192 drugs) contained in the databases and their corresponding target data.

IV. Examining Whether the Target Genes of the Drug are Significantly Targeted to the Key Gene List by Statistical Analysis

The Kolmogorov-Smirnov test was used to see if the target genes of the drug were significantly distributed at the top of the key genes list obtained in step II, that is, whether the target genes of the drug were targeted to the most important genes in the pathogenesis of disease in the patient. The smaller the statistical p-value was, the more suitable the drug was for this patient.

V. Predictive Performance Assessment: Performing Statistical Analysis on the Efficiencies of the Successfully-Listed and Currently-Under-Development Drugs of Alzheimer's Disease

Statistical analysis was performed on the drug efficiencies of the successfully-listed and currently-under-development drugs of Alzheimer's disease and P values were obtained by the Kolmogorov-Smirnov test. The calculation method of efficiency was: if the P value of the KS test was less than 0.05/0.01, then the drug was considered effective for the patient, and the proportion of effective patients in the patient population was analyzed.

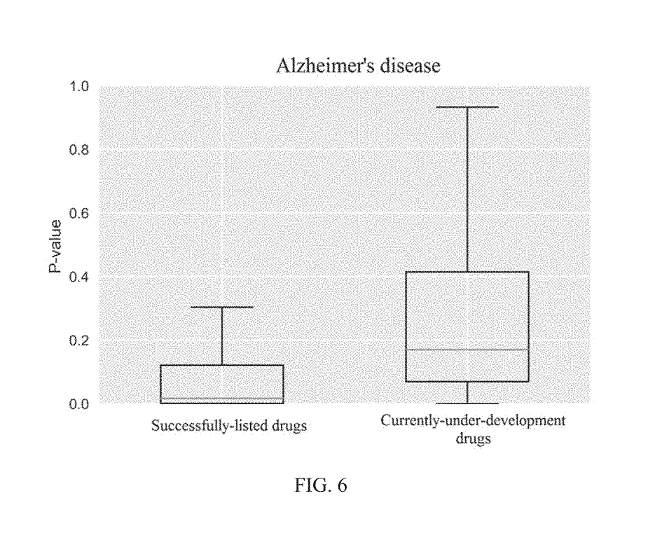

The Wilcoxon rank-sum test was used to test whether there was a significant difference in the targeting efficiencies (The P-value obtained from the Kolmogorov-Smirnov test) of the two groups of drugs towards the patient, and found that the statistical P value was 0 (The P distribution of KS tests for the two groups of drugs was shown in FIG. 6). Meanwhile, the Wilcoxon rank-sum test was also used to calculate the cumulative effective rate of the two groups of drugs in the patient population and found that the P-value of the difference between the efficiencies of the two groups of drugs was 0.0012. Our method confirmed that in Alzheimer's disease, the drugs successfully listed were significantly more effective than the drugs currently under development in the human population.

### Embodiment 5: Use the Method of the Present Invention for Personalized Medicine Screening for Patients with Specific Subtypes of Breast Cancer

I. Obtaining Gene Expression Data of Breast Cancer Patient Samples, Gene Expression Data of Control Samples, and Subtype Information of Each Patient

This step was the same as step I of embodiment 3, the difference was that the subtype information of these 1109 breast cancer patients was obtained.

Calculating the key genes list in the pathogenesis of disease in patients.

This step was the same as step II of embodiment 3.

II. Obtaining Drug (Combination) Data

Drug target databases (including DrugBank: http://www.drugbank.ca/, TTD: http://bidd.nus.edu.sg/group/ttd/ttd.asp and ClinicalTrials: https://www.clinicaltrials.gov/) were searched in order to retrieve all the drugs contained in the databases and their corresponding target data. Only the listed drugs with activities for cancers similar to breast cancer, such as ovarian cancer, prostate cancer, etc., were selected as candidate drugs against certain subtypes of breast cancer. Chemotherapy drugs for breast cancer that were commonly used clinically were in turn paired with the above-mentioned candidate drugs to act as candidate drug combinations against certain subtypes of breast cancer.

III. Obtaining Target Gene Data of the Above-Mentioned Drugs

This step was the same as step III of embodiment 1.

IV. Calculating the Drug Efficacy of Each Drug (Combination) for Each Patient

This step was the same as step IV of embodiment 1.

V. Personalized Medicine Screening for Patients with Specific Subtypes of Breast Cancer

According to the results of step IV, data of drugs with significant p-values (in the present embodiment, p value<0.05 was considered as significant) against patient numbers of different subtypes was collected. If the effective rate of a certain drug (combination) was significantly higher than its effective rate in all breast cancer patients, then the drug (combination) is considered as a suitable drug (combination) for patients with this subtype. A breast cancer subtype significantly suited to a certain drug (combination) was found out by a statistical test (a binomial distribution test method was employed in the present embodiment). A more significant test indicated that the drug (combination) was more suited to the corresponding subtype. The personalized medicine screening results for patients with specific subtypes of breast cancer were recorded in Table 1. The results showed that 95% of single-component drugs and 56% of drug combinations have breast cancer-related clinical or literary support to prove the effectiveness of the method.

TABLE 1
screening results of the suitable drugs (drug combinations) for patients
with certain subtypes of breast cancer (p-value <0.01)

| | | Number of drugs (drug | |
|---|---|---|---|
| | | combinations) | |
| | | associated with breast | |
| | Number of | cancer in clinical | |
| | drugs (drug | records or literature | |
| Types | combinations) | reports | Percentage |
| single composition | 20 | 19 | 95% |
| drug | | | |
| Drug combinations | 476 | 267 | 56% |

What is claimed is:

1. A systematic pharmacological method for personalized medicine, characterized in that it comprises the following steps:
Step 1: obtaining high-throughput biological data to build a biological network, the biological network describes interactions between genes;
Step 2: obtaining gene expression data of a diseased tissue of a specific patient suffering from a disease and the corresponding control data, calculating a differential expression value for genes of the specific patient accordingly;
Step 3: sorting genes of the specific patient according to their importance using a gene rank algorithm according to the biological network and the differential expression value of genes of the specific patient, selecting a plurality of top-ranked, more important genes as key genes to construct a key genes list;
Step 4: obtaining a target gene corresponding to a drug to be tested according to information in a drug target database; and
Step 5: using statistical analysis to check whether the target gene of the drug to be tested is significantly targeted to the key genes list in order to predict the activity of the drug on the specific patient; screening a drug suitable for the specific patient to achieve personalized medicine for the specific patient.

2. The systematic pharmacological method for personalized medicine according to claim 1, characterized in that the biological network is a directed network or an undirected network; the directed network is a gene dependency network; the undirected network is a protein interaction network or a gene co-expression network.

3. The systematic pharmacological method for personalized medicine according to claim 2, characterized in that the gene dependency network is constructed by obtaining gene expression data and the corresponding clinical phenotype data for diseased tissues of a plurality of patient samples corresponding to the disease, and comprises the following steps:
Step A. using the gene expression data and the corresponding clinical phenotype data, obtaining a gene dependency index between any two genes; and
Step B. setting a threshold value, obtaining significantly dependent gene pairs, constructing all the significantly dependent gene pairs into the gene dependency network;
in the gene dependency network, a vertex is a gene, a directed edge between two vertices indicates the presence of a significant dependency relationship between an end-point gene and a relationship between a start-point gene and the disease phenotype.

4. The systematic pharmacological method for personalized medicine according to claim 2, characterized in that the protein interaction network is constructed by obtaining protein interaction pairs with high human confidence from a protein interaction database, and comprises the following steps:
Step I. downloading human protein interaction data from the protein interaction database; and
Step II. screening the protein interaction pairs with high confidence, and constructing all the protein interaction pairs with high confidence as the protein interaction network;
in the protein interaction network, a vertex is a gene, an edge between two vertices indicates an interaction between two proteins corresponding to the two vertices.

5. The systematic pharmacological method for personalized medicine according to claim 3, characterized in that the gene-dependency index is conditional mutual information, the conditional mutual information is calculated as follows:
CMI(A,P|B)=I_{high}(A,P)−I_{low}(A,P)
where CMI (A,P/B) denotes mutual information between a gene A and a disease phenotype P under the condition of a gene B; I_{high}(A,P) denotes mutual information between the expression value of the gene A and disease phenotype data in patient samples with highly expressed gene B; I_{low}(A,P) denotes mutual information between the expression value of the gene A and disease phenotype data in patient samples with lowly expressed gene B;
the mutual information is calculated as follows:
$$I ( A , P ) = {\sum }_{A} {\sum }_{P} p ( A , P ) {log}_{2} \frac{p ( A , P )}{p ( A ) p ( P )}$$
where p(A) is the probability of a variable A, p(A,P) is the joint probability of the variable A with a variable P, the variable A is the expression value of the gene A, the variable P is the disease phenotype P.

6. The systematic pharmacological method for personalized medicine according to claim 1, characterized in that the gene expression data is obtained by a gene expression analysis method, the gene expression analysis method includes at least one of gene chip or RNA-Seq, the differential expression value for genes of the specific patient is obtained by fold-change.

7. The systematic pharmacological method for personalized medicine according to claim 1, characterized in that the gene rank algorithm is a revised PageRank algorithm, the formula of the revised PageRank algorithm is as follows:
$${r}_{j}^{n} = ( 1 - d ) {ex}_{j} + d {\sum }_{i = 1}^{N} \frac{{w}_{ij} {r}_{i}^{n - 1}}{{deg}_{i}}$$
where rⱼⁿ is a calculated importance value of a gene j, exⱼ is an initial value of the gene j, and wᵢⱼ is a relationship between genes in the biological network;
when the biological network is a directed network, if a gene i depends on the gene j, then wᵢⱼ=1, otherwise wᵢⱼ=0; when the biological network is an undirected network, if there is interaction between the gene i and the gene j, then wᵢⱼ=1 and wⱼᵢ=1, otherwise wᵢⱼ=0 and wⱼᵢ=0; rᵢⁿ⁻¹ is a value of the i^{th} gene after the (n−1)^{th} iteration; deg, is an out-degree of a vertex i; parameter d (0≤d<1) is a constant and d denotes a proportion of the biological network during calculation.

8. The systematic pharmacological method for personalized medicine according to claim 1, characterized in that the drug target database is DGIdb, TTD, and Drugbank, the target gene corresponding to a drug to be tested is a union set of target gene data of three databases including DGIdb, TTD, and Drugbank.

9. The systematic pharmacological method for personalized medicine according to claim 1, characterized in that the statistical analysis is enrichment analysis; by analyzing whether the target gene corresponding to a drug to be tested is enriched in the key genes list, whether it significantly targets the important genes list is determined.

10. The systematic pharmacological method for personalized medicine according to claim 9, characterized in that an enrichment analysis model used in the enrichment analysis is the Kolmogorov-Smirnov test.

11. The systematic pharmacological method for personalized medicine according to claim 1, characterized in that the disease is cancer, the diseased tissue is cancer tissue, and the control data is the gene expression data of paracarcinoma tissue or normal tissue.

12. Application of the systematic pharmacological method for personalized medicine according to claim 1 in the screening of personalized drugs and/or drug combinations, and in personalized medicine.
